(19) United States
(12) Patent Application Publication
(10) Pub. No.: US 2020/0222907 A1
(43) Pub. Date: Jul. 16, 2020

(54) SYSTEMS AND METHODS FOR DETERMINING PROBATIVE SAMPLES AND ISOLATION AND QUANTITATION OF CELLS

(71) Applicants: DxNow, Inc., Gaithersburg, MD (US); The Brigham and Women's Hospital, Inc., Cambridge, MA (US); Board of Trustees of the Leland Stanford Junior University, Redwood City, CA (US)

(72) Inventors: Leonard Klevan, Cave Creek, AZ (US); Fatih lnci, Palo Alto, CA (US); Utkan Demirci, Stanford, CA (US)

(21) Appl. No.: 16/824,228

(22) Filed: Mar. 19, 2020

Related U.S. Application Data
(63) Continuation-in-part of application No. 15/522,232, filed on Apr. 26, 2017, now abandoned.

(51) Int. Cl. B01L 3/00; C12M 1/34; C12M 1/36; C12M 3/06; C12M 1/42

(52) U.S. Cl. CPC B01L 3/502761; C12M 41/36; B01L 2200/027; C12M 23/16; C12M 35/06; C12M 41/48

(57) ABSTRACT

Embodiments of the present disclosure relate to a magnetic bead platform for isolating sperm cells from biological samples. In some embodiments, such magnetic bead platforms integrate recognition reagents to its surface to bind target cells, such as sperm cells. Such embodiments provide the ability to at least one of rapidly isolate and quantitate sperm cells from biological samples as occur in sexual assault evidence, for example, thereby enhancing identification of suspects in these cases and contributing to the safety of society.

### CROSS REFERENCES TO RELATED APPLICATIONS

The present application is a continuation-in-part of U.S. Ser. No. 15/522,232, filed Sep. 30, 2015; which claims benefit pursuant to 35 U.S.C. § 371 to PCT Application No. 2015053370, filed Sep. 30, 2015; and which claims the benefit under 35 U.S.C. § 119(e) of U.S. Provisional Application Ser. No. 62/058,072, filed Sep. 30, 2014. The entire contents of the above-referenced patents and patent applications are hereby expressly incorporated herein by reference.

### GOVERNMENT RIGHTS

This invention was made with Government support under award no. 1464673 awarded by the National Science Foundation. This invention was also made with U.S. Government support under DOJ Award No: 2017-NE-BX-004 awarded by the Department of Justice. This invention was also made with support under NIJ Award No: 2019-NE-BX-0003 awarded by the National Institute of Justice. The Government may have rights in and to the presently disclosed and/or claimed inventive concepts.

### FIELD OF THE INVENTION

The present disclosure relates to platforms for at least one of capturing, identifying and studying biological materials, and more particularly, to microfluidic channel platforms (for example) for detecting and/or identifying samples containing sperm cells, and isolating and analyzing captured sperm cells for DNA analysis (for example). In some embodiments, such microfluidic platforms integrate imaging technology. Such embodiments provide the ability to at least one of rapidly isolate and quantitate sperm cells from biological mixtures as occur in sexual assault evidence, for example, thereby enhancing identification of suspects in these cases and contributing to the safety of society.

### BACKGROUND

Although forensic DNA testing has contributed immensely to the successful processing and analysis of evidence materials collected at crime scenes, especially crimes of a sexual nature, the time-consuming steps involved in such processes and analysis have led to an immense backlog that has overwhelmed the available capacity of forensic laboratories. For example, it currently takes hours to separate sperm cells from samples, and in some cases, the effort may be a waste of precious resources if the sample does not contain usable cells. Such situations arise due to the lack of reliable methods for identifying useful samples prior to the extraction of the sperm cells.

### SUMMARY OF SOME OF THE EMBODIMENTS

Some embodiments of the present disclosure address problems of the prior art. For example, in some embodiments, a system for capturing and/or detecting target cells in a biological sample are provided and include (for example): one or more microfluidic channels for receiving a biological sample, a recognition reagent linked to a surface of the one or more channels, which may also be referred to as a capture molecule or material that may be linked to the surface of a channel (or other surface, e.g., bead) directly or via another molecule and/or substance (e.g., the term “reagent” or phrase “recognition reagent” can correspond to or be referred to as a capture molecule or material, or similar functionality). The reagent is configured to capture one or more target cells contained in the biological sample by binding with the one or more target cells, and a monitoring means configured to at least one of monitor the surface and detect one or more captured target cells bound with the reagent linked to the surface. The monitoring means can be configured to at least one of receive and collect data on the captured target cells. In some embodiments, the monitoring means may comprise an imaging means configured to acquire images of captured target cells. Additionally (or in place of), such monitoring means may include at least one of mechanical means, electrical means, optical means, photonic means, and plasmonic means. Further, the monitoring means may correspond to or include a smartphone for at least one of image capture and information/image analysis. For example, in some embodiments, the smartphone is equipped with an application configured for receiving and/or collecting data of at least one of the surface (e.g., image information) and data on any captured target cells. In some embodiments, the target cells in the biological sample can be sperm cells, blood cells, bacteria, yeasts, fungi, and/or viruses.

In some embodiments, the recognition reagent comprises an oligosaccharide sequence. The oligosaccharide sequence may comprise a sialyl-Lewis^{x} oligosaccharide sequence. In some embodiments, the microfluidic channels can have dimensions ranging from about 25 micron to about 80 micron.

Some embodiments of the current disclosure are directed to (or further include) methods for capturing and/or detecting target cells in a biological sample. Such methods comprise: providing a surface having linked thereto one or more oligosaccharide molecules, where the oligosaccharide molecules are configured to capture one or more target cells, exposing the surface to a biological sample, capturing one or more target cells contained in the sample, where a target cell is captured by binding with at least one of the oligosaccharide molecules, and at least one of monitoring the surface and detecting the at least one captured target cell. In addition, the steps may further comprise at least one of receiving and collecting data corresponding to at least one of the surface and captured target cells. Further, the steps may include at least one of releasing, lysing, and processing the captured target cells. In some embodiments, the target cells may be sperm cells, blood cells, bacteria, yeasts, fungi, and/or viruses.

In some embodiments, the at least one of monitoring and detecting step may comprise receiving and/or collecting data associated with at least one of the surface and captured target cells bound thereon via the oligosaccharide. For example, the data may be received and/or collected via a monitoring means, and the at least one of receiving and collecting data may comprise imaging the surface and/or bound target cells. In some embodiments, the oligosaccharide molecules may comprise sialyl-Lewis^{x} oligosaccharide molecules. In some embodiments, the step of exposing comprises flowing the biological sample over the surface. The surface may include at least one of the inner surface of one or more microfluidic channels and the surface of one or more beads.

Some embodiments of the current disclosure also include a method for capturing and detecting target cells in a plurality of biological samples, comprising: identifying, via shadow imaging (for example), probative samples for capturing and/or detecting target cells from the plurality of biological samples, providing a surface having linked thereto one or more oligosaccharide molecules, where the oligosaccharide molecules are configured to capture one or more target cells, exposing the surface to the probative samples for capturing and detecting target cells, capturing one or more target cells contained in the probative sample, where a target cell is captured by binding with at least one of the oligosaccharide molecules, and extracting DNA of the target cells contained in the probative sample.

One of skill in the art will appreciate that some embodiments may be configured such that, target cells can be selectively separated from a sample (e.g., for enrichment), and, in some embodiments, non-target cell types can be separated so as to eliminate them from the sample.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings are primarily for illustrative purposes and are not intended to limit the scope of the inventive subject matter described herein. Moreover, the drawings are not necessarily to scale, as, in some instances, various aspects of inventive subject matter may be shown exaggerated or enlarged to facilitate an understanding of different features. Additionally, in the drawings, like reference characters generally refer to like features (e.g., functionally similar and/or structurally similar elements).

FIG. 1 shows an example flow diagram for identifying samples containing sperm cells, and for isolating and analyzing captured sperm cells, according to some embodiments.

FIGS. 2A-B show schematic illustrations of complementary metal-oxide semiconductor (CMOS)-integrated (FIG. 2A) and smartphone-integrated (FIG. 2B) microfluidic systems for shadow imaging, capturing, and analyzing sperm cells, according to some embodiments.

FIG. 3A shows the monitoring, via shadow imaging, of sperm cells captured and isolated in a microfluidic platform, according to some embodiments.

FIG. 3B shows example microscope images of various types of sperm cells, and identification thereof, according to some embodiments.

FIGS. 4A-B show an example microfluidic device with microchannels for selective sperm capture, isolation, detection and quantification, according to some embodiments.

FIG. 5 shows an example detailed view of the capture of sperm cells utilizing sialyl-Lewis^{x} sequence (SLeX) oligosaccharide, according to some embodiments.

FIG. 6A shows a schematic illustration of capture of sperm cells in the microchannels of a microfluidic device in the presence of a blocking agent, according to some embodiments.

FIGS. 6B-C illustrate the effect of a blocking agent and SLEX concentration in the sperm capture efficiency of a microfluidic device, according to some embodiments.

FIG. 7 shows an aspect of surface chemistry for capture of sperm cells utilizing sialyl-Lewis^{x} sequence (SLeX) oligosaccharide, according to some embodiments.

FIGS. 8A-B show example results of differential extraction of sperm and/or epithelial cells, according to some embodiments.

FIGS. 9A-D show an example differential extraction process of aged sperm cells to isolate various types of sperm cells from epithelial cells, according to some embodiments.

FIG. 10 is a flow diagram of a non-limiting embodiment of the workflow and surface chemistry procedure for sperm isolation utilizing SLeX conjugated magnetic beads constructed in accordance with the presently disclosed and/or claimed inventive concept(s).

FIGS. 11A-11B are diamond-shaped Box-Whisker plots showing sperm capture efficiency for increasing concentrations of 4-aminobenzoic acid hydrazide (4-ABAH) (FIG. 11A) and increasing concentrations of SLeX oligosaccharide (FIG. 11B).

FIGS. 11C-11D are fluorescence microscope images of captured sperm cells stained with 4′,6-diamidino-2-phenylindole (DAPI). FIG. 11C is a brightfield fluorescent image while FIG. 11D is a non-brightfield fluorescent image.

FIG. 12 is a bar graph showing the dynamic light scattering (DLS) properties and measurements associated with the particle size of the magnetic beads.

FIG. 13 is a graphical representation showing the Fourier transform infrared (FTIR) spectroscopy measurements of NHS-activated magnetic beads and SLeX-modified magnetic beads, both of which are constructed in accordance with the presently disclosed and/or claimed inventive concept(s).

FIGS. 14A-14C are diamond-shaped Box-Whisker plots showing sperm capture efficiency for increasing magnetic bead counts (FIG. 14A), increasing incubation times (FIG. 14B), and increasing sperm cell counts (FIG. 14C).

FIG. 15 is a comparative bar graph showing the capture efficiency of sperm cells and buccal epithelial cells when utilizing non-modified beads, for example, NHS-activated magnetic beads and modified-beads, for example, SLeX conjugated magnetic beads, constructed in accordance with the presently disclosed and/or claimed inventive concepts(s).

### DETAILED DESCRIPTION OF SOME OF THE EMBODIMENTS

Embodiments to a sperm cell capture system and methodology (together or separately referred to as “platform(s)” or “system(s)”) for direct and intact sperm cell detection and/or isolation using the inner surface(s) microfluidic channels are disclosed herein. In some embodiments, the platforms provide expedited testing for forensic as well as hospital and primary care settings. Moreover, in some embodiments, label-free, bio-detection functionality such as electrical, mechanical and optical mechanisms (including photonic and plasmonic) can be used for the monitoring, detection, capture, isolation, and/or quantification of sperm cells from bodily and clinically relevant fluids. In some embodiments, conjugated magnetic beads may be used (in addition to or in place of the surfaces of microfluidic channels) for capturing sperm cells. In some embodiments, detection for multiple morphologies of sperm cells ranging from forensic applications to laboratory research, medical diagnostics and drug development/treatment are provided.

In some embodiments, a detection method is provided and includes flowing a biological sample within one or more microfluidic channels so as to capture sperm cells and perform shadow imaging using, for example, holographic algorithms (also including other static and dynamic imaging algorithms) with the microfluidic platform to obtain one or more images of bound sperm cells (for example). Some embodiments of holographic imaging are discussed in the publication by Sobieranski et al., Light: Science & Applications 4, e346; doi:10.1038/lsa.2015.119 (2015), entitled “Portable Lensless Wide-field Microscopy Imaging for Health-Care Applications using Digital In-line Holography and Multi-Frame Pixel Super-Resolution,” the content of which is incorporated herein by reference in its entirety.

In some embodiments, a plurality of capture reagents may be used to isolate target particles (e.g., cells, molecules) and/or target analytes from forensic samples containing such target particles. In particular, and for example, embodiments of microchips (e.g., forensic microchips) described herein may be used to capture target cells/analytes with high efficiency and specificity. Surfaces having the captured cells/analytes can then be monitored under, for example, an optical shadow imaging means which may be used with one or more algorithms such as holographic algorithms as well as other static and dynamic imaging algorithms for label-free detection and quantification. In some embodiments, the combination of such microchip embodiments with a lens-less imaging system (i.e., shadow imaging) provides for a portable and optionally battery-powered capture and detection system which can be used I the field (for example). Such embodiments are configured to overcome many of the deficiencies with existing technologies, which are limited by required equipment, time, cost, and other processing factors. In come embodiments, a shadow imaging platform integrated with a microfluidic device is provided, which includes an inlet for reception of a biological sample. The inlet is in fluid communication with one or more microfluidic channels, each having at least one surface configured for capture detection with one or more capture reagents.

FIG. 1 is an example flow diagram for at least one of identifying samples containing sperm cells, and isolating and/or analyzing the captured sperm cells. Initially, samples which are hoped to contain biological materials (which may be referred to as a biological sample according to embodiments of the disclosure) from evidence material may be extracted using several methods. For example, pieces of evidence material samples (e.g., cotton swap or gauze pieces) may be eluted in phosphate buffered saline (PBS) (e.g., 500 μL of 1×PBS) and placed in a low temperature mixer (e.g., 4° C. thermomixer) set at a high rpm (e.g., about 1000 rpm) for a set amount of time (e.g., about 1 hour). The pieces may then be removed and placed in spin baskets that are subsequently centrifuged for short period of time (e.g., about 5 minutes) to pellet the solids in the solution. Some of the 1×PBS (about 300 μL) may then be removed without disturbing the pellet, which may be suspended by pulse vortexing.

After obtaining one or more biological samples, at step 101, such samples suspected of containing target cells (e.g., sperm cells) may be screened to identify probative samples that are candidates for further analysis (for isolating and study sperm cells contained within the samples). Such embodiments are highly advantageous compared to prior devices/methodologies where only few of collected samples are screened due to the long period of time to perform DNA testing. Prior devices/methodologies are often a waste of time and resources as most samples do not contain viable sperm cells for analysis.

Thus, in sharp contrast to the prior art, some of the embodiments of the disclosure allow for preliminary testing of samples (and in some embodiments, at the crime scene or hospital) for the presence of sperm. Such embodiments, include, for example, a rapid imaging via a microfluidic chip/cartridge that initially detects the presence of sperm to identify the probative samples for further analysis (e.g., DNA analysis). An exemplary method embodiment of such detection includes inputting the sample (or a portion thereof) suspected of containing sperm cells into a microfluidic platform. After the sample/portion thereof is positioned (e.g., flowed) within the one or more channels, imaging may be performed (i.e., shadow imaging) on the sample/portion to ascertain whether the sample contains sperm cells. Other methods of detecting presence of sperm cells include direct microscope analysis, use of chemical stains, and/or the like. Details on at least the use of staining methods are discussed in Allery et al., J. Forensic Science 46(2): 349-351 (2001), entitled “Cytological Detection of Spermatozoa: Comparison of three staining methods,” the content of which is incorporated herein by reference in its entirety.

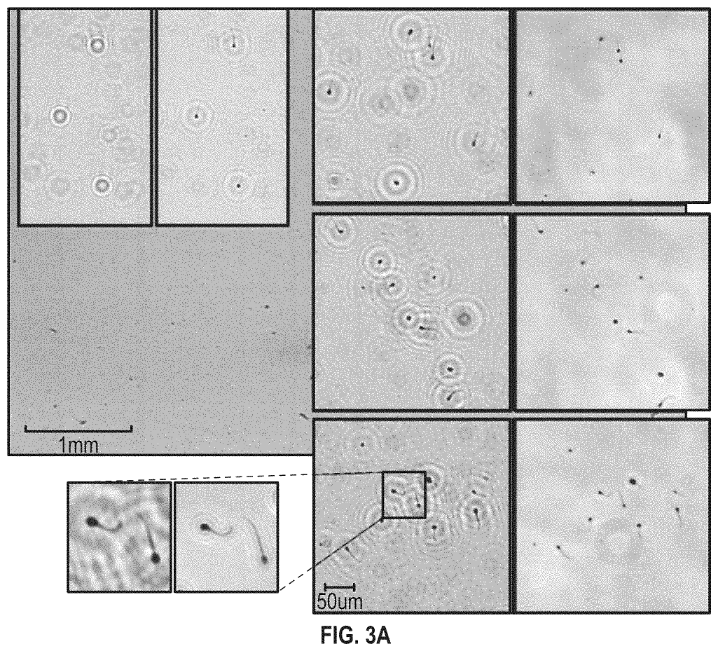

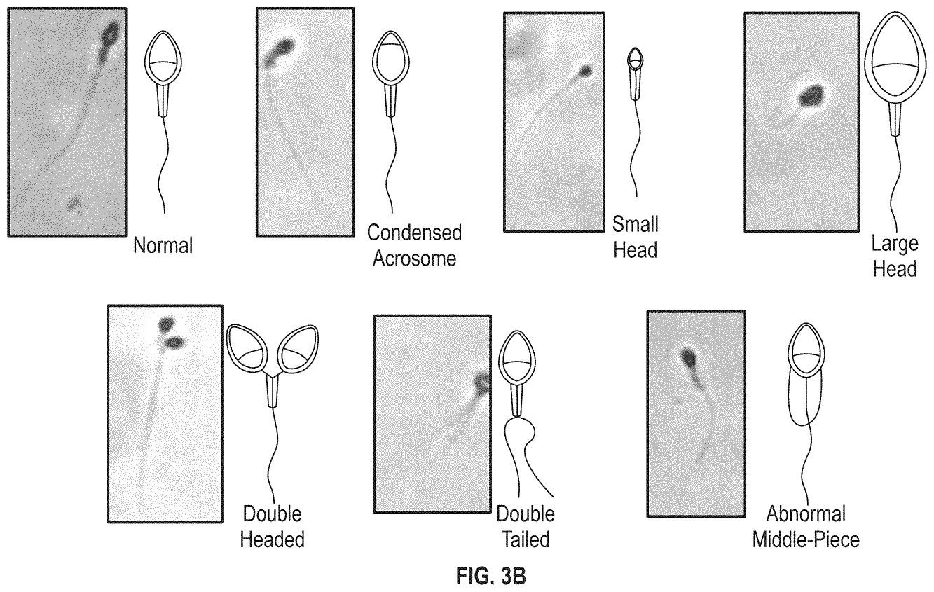

In some embodiments, for example, the imaging comprises shadow imaging. Such shadow imaging can utilize holographic, static and/or dynamic algorithms so as to help obtain images of sperm cells in the sample/portion. In some cases, such imaging can not only identify sperm cells, but also provide further details on captured sperm cells, such as but not limited to, the quantity of the sperm cells, which may allow for the determination of the more probative samples that can be used for additional focused DNA testing. For example, the imaging may provide a broad range of different morphologies of sperm cells. In some embodiments, other imaging techniques may also be used. For example, microscope imaging may be used to obtain a broad range of different morphologies of sperm cells as presented in FIG. 3B. Analysis of these images may allow a laboratory technician to identify the above noted probative samples.

Upon the selection of the probative samples for DNA analysis, in some embodiments, the same or new (and/or different) microfluidic platform may be utilized to extract biological components (step 102) from the samples, thereafter, sperm cells are captured (step 103). Such captured sperm cells can then be further analyzed (step 104) for DNA analysis (for example).

In some embodiments, the extracted biological components may now contain epithelial as well as sperm cells, and one may desire to isolate the sperm cells for analysis, e.g., step 103. For example, for samples derived from crime scenes such as sexual assaults, sperm cells from a perpetrator, epithelial cells primarily from the victim but also some from the perpetrator, and perhaps some DNA resulting from lysed cells may occur in the biological components. Depending on the specifics of the case, there may be multiple contributors of the epithelial and sperm cells. In some embodiments, the epithelial cells may be lysed, and the resulting mixture may flow through the device, which may result in the collection of sperm cells present and accounting of the sperm cells by a holographic imaging system.

In some embodiments, the lysis of the epithelial cells may occur after the capture of the sperm cells on the microfluidic surface or any solid surface to which the capture moiety has been linked. The lysed or unlysed epithelial cells along with free DNA and other components of the biological sample may then be collected and could be retained if there is a desire to analyze the DNA of the material based on the specifics of the forensic case.

At step 104, in some embodiments, the sperm cells are now separated and purified from the other components in the biological components, and the sperm cells may be eluted from the microfluidic platform (or bead or micro titre plate or any insoluble substrate) if there is a reversible linker present in the sperm attachment moiety or the sperm may be lysed on the substrate to release the DNA which can then be isolated for subsequent analysis.

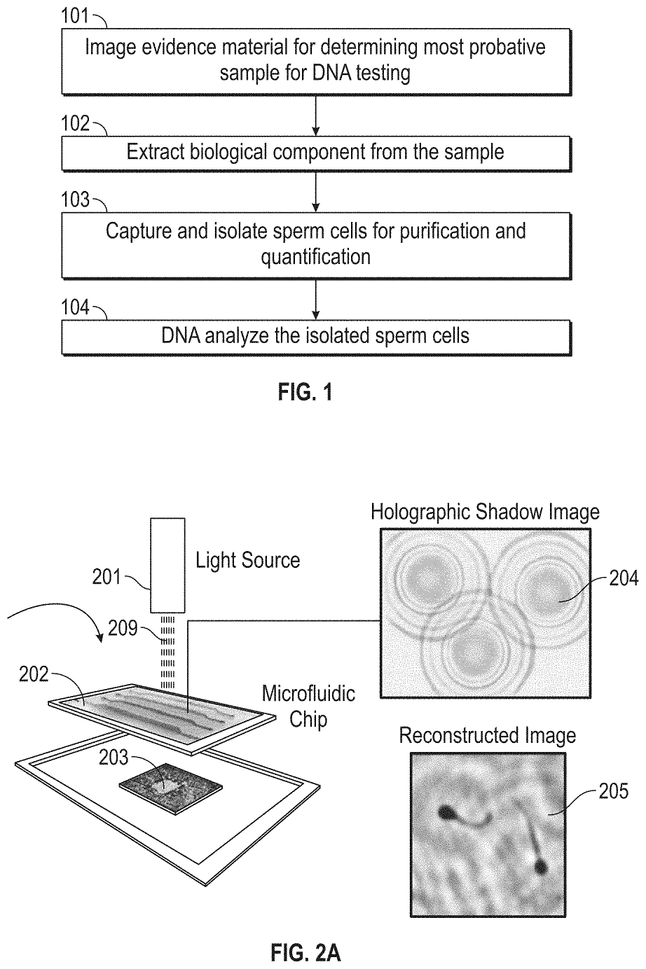

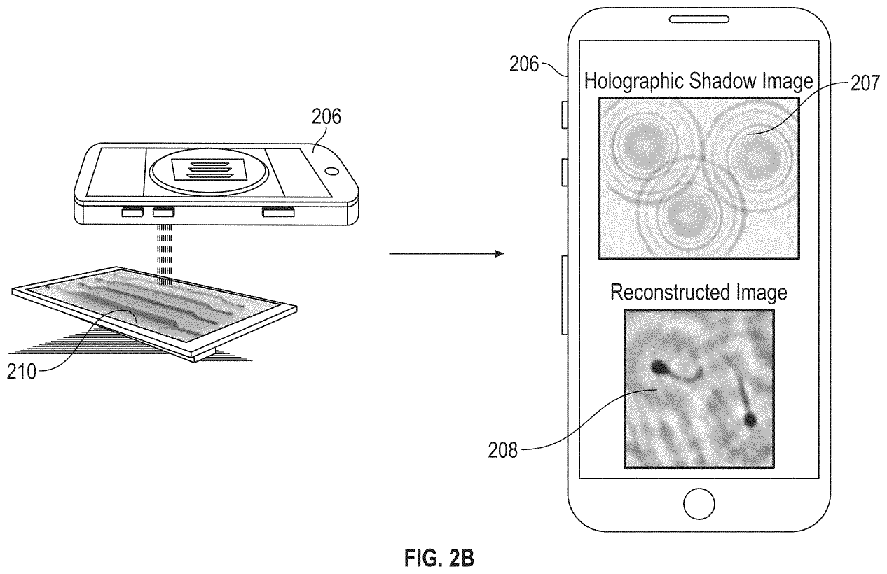

With reference to FIG. 2, in some embodiments, shadow imaging means (configured such that it does not require pre-labeling of a sample) can be utilized for sperm cell imaging/visualization. In some embodiments, a microfluidic chip is provided which includes a sperm capturing reagent such as, but not limited to, sialyl-Lewis^{x} sequence (SLeX), which may be employed along. The imaging means (e.g., shadow imaging detector) can be integrated with different algorithms such as holographic as well as other static and dynamic imaging algorithms. Moreover, in such embodiments, the imaging means may also include LED illumination and a CMOS image sensor. The reagent is configured to capture (and thus, separate) sperm cells from epithelial cells (e.g., in sexual assault evidence). Such a microfluidic process can also allow for quantification of sperm cells bound to a channel(s) in the chip, and thus, can be used to identify the probative samples themselves (and effective analytical methods for forensic analysis thereafter).

FIGS. 2A-B provide schematic illustrations of complementary metal-oxide semiconductor (CMOS)-integrated (FIG. 2A) and smartphone-integrated, for example (FIG. 2B), microfluidic systems that can be used to at least one of initially identify the probative samples, and/or to shadow image, capture, and/or analyze sperm cells contained in the samples. In FIG. 2A, light 209 from a light source 201 may be shone onto a microfluidic chip/cartridge 202 with CMOS image detector. The cartridge/chip comprises microfluidic channels upon which probative samples are provided therein (e.g., via flow) that include sperm cells. In some embodiments, a shadow image 204 (e.g., holographic) of the sperm cells contained in the probative samples may be obtained by via the CMOS image sensor (or other sensor; e.g., CCD sensor). The holographic shadow image 204 may further be processed (e.g., via holographic, static, dynamic, and/or the like imaging algorithms) to produce a reconstructed image 205 of the sperm cells in the samples. Features of this shadow imaging means (which are generally lens-less) have been discussed in the article by Zhang et al., entitled “Lensless imaging for simultaneous microfluidic sperm monitoring and sorting,” in the publication Lab on a Chip, issue 15, vol. 11, pp. 2535-2540 (2011), and in PCT Publication No. WO/2014/047608, entitled “Portal and Method for Management of Dialysis Therapy,” the entire contents of both of which are expressly incorporated by reference herein.

In some embodiments, in place of or in addition to a microfluidic cartridge/chip-based shadow imaging system, a smartphone-integrated microfluidic system may be used for studying the probative samples. FIG. 2B shows a smartphone 206 capturing data (e.g., image) from a microfluidic cartridge/chip 210 comprising a sample that contains sperm cells. For example, if the smartphone contains a CMOS chip, then the smartphone may be attached to the shadow imaging device to record the image of sperm cells. In some embodiments, the image data may include information that allows an application operating on the smartphone to identify the sperm cells and some or all of the associated properties thereof. For example, the data may include contrast, color, sharpness, hue, shadow etc., information that allows the application to determine the type, size, etc., of the sperm cells being studied (as well as the number of sperm cells). Accordingly, from such data, in some embodiments, the application can produce a shadow image 207 (e.g., holographic) from which a reconstructed image 208 of the sperm cells can be created. For example, images obtained by the smartphone may be analyzed using holographic algorithms (e.g., a holographic software) to determine the presence or absence and in some cases quantity of sperm cells in the sample. In some embodiments, the images may not be collected by the smartphone, but they may be received by an external server that, by using the holographic algorithms, processes the images so as to determine the presence/absence and additionally quantity of the sperm cells in the samples. Further, the results may be received by the smartphone (e.g., via wired or wireless (e.g., wifi, Bluetooth, etc.) connections) from the server. The use of a smartphone is particularly beneficial in that the initial screening of evidence materials to determine a probative sample for further analysis and DNA testing (or inclusion into a rape kit, depending on the circumstance) can be performed on location (e.g., at a crime scene, hospital, etc.) relatively rapidly and conveniently given the portability of smart phones (e.g., compact, mobile computing/imaging devices).

FIG. 3A shows shadow images of sperm cells captured and isolated in a microfluidic platform, according to some embodiments. These images allow for the monitoring of the captured sperm cells, allowing one to identify a broad morphology of sperm cells with applications not only in DNA forensics but also in laboratory research, medical diagnostics, drug development/treatment, and/or the like. For example, from the study of shadow images such as the ones depicted in FIG. 3A, in some embodiments, one may identify several forms of sperm cells, including normal sperm cells and sperm cells with condensed acrosome, small heads, large heads, double heads, doubled tails and an abnormal middle-piece, e.g., FIG. 3B.

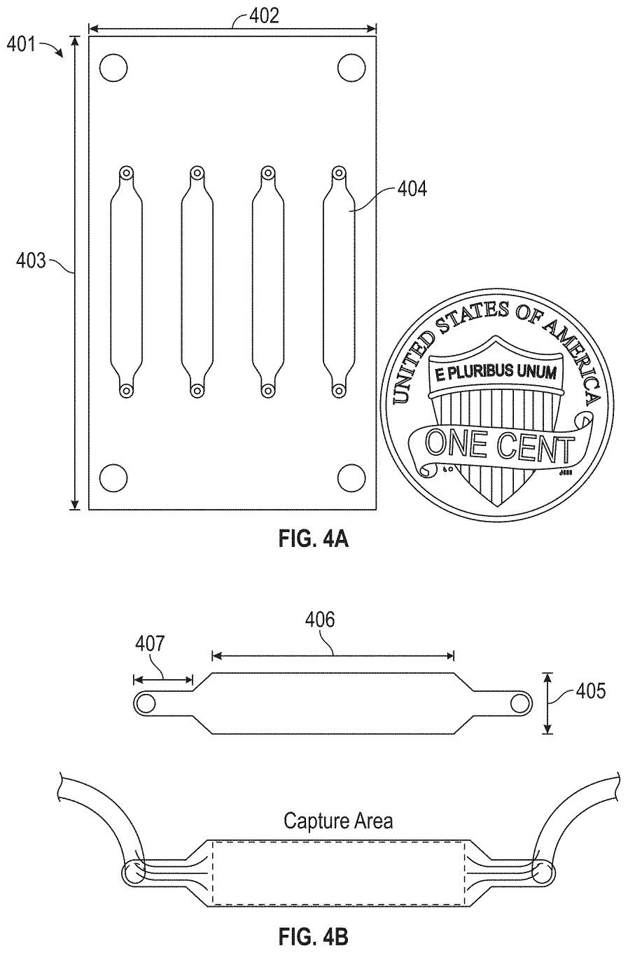

With reference to FIG. 4A, in some embodiments, a microfluidic device 401 for selective sperm cell capture, isolation, detection and quantification is shown (at least one thereof). In some embodiments, the device 401 may be fabricated without utilizing photolithographic methods or a clean room. The device 401 may be constructed so as to have a plurality of microfluidic channels 404 (one or more). For example, as shown in the embodiment of FIG. 4A, the microfluidic device 401 may include four parallel microfluidic channels within an area measuring about 40 mm in length 403 and about 24 mm in width 402.

FIG. 4B provides a schematic diagram of a microfluidic channel 404 comprising three regions, an inlet and an outlet, e.g., 407, and a capture area 406 where the capture and isolation of the sperm cells take place. In some embodiments, the capturing of sperm cells in the capture area 406 is facilitated by the differences in the dimensions of the lateral diameter 405 of the microchannel 404 and that of the inlet and/or the outlet 407. For example, the lateral diameter 405 may measure about 2.5 mm while that of the openings may measure about 1.53 mm. Further, the much larger length of the capture area (e.g., about 13.5 mm) also facilitates the capture and isolation of the sperm cells.

To construct the device, in some embodiments, poly(methyl methacrylate) (PMMA) (1.5 mm thick, McMaster Carr, Atlanta, Ga.) and double-sided adhesive (DSA) film (80 μm thick, iTapestore, Scotch Plains, N.J.) are fabricated using a laser cutter (Versa Laser™ Scottsdale, Ariz.). The inlets and outlets 407 at each end of the channels 404 are configured on the PMMA layer, and glass cover slips can then assembled using the DSA. To clean the chip base, the glass cover slip can be sonicated for about 15 min in ethanol. Following the cleaning step, the cover slip is then washed with distilled water and dried under nitrogen gas. To modify the surface, both sides of the glass cover slip can be plasma-treated for about 90 seconds. Then, PMMA, DSA, and glass cover slip can be assembled to produce the microfluidic device.

In some embodiments, the substrate of the sperm capture area/region can be optically transparent to facilitate shadow imaging and optical measurement. Thus, polystyrene, glass parylene, quartz crystal, graphene and mica layers, and poly(methyl methacrylate) can be used for the substrate. These materials are optically transparent and are capable of supporting the functionalization of the surfaces of the capture area 406, which selectively bind to sperm cells via surface recognition elements linked thereto (e.g. reagent) such as specific saccharides units and antibodies and which possess the optical properties for the monitoring of the binding and capture events.

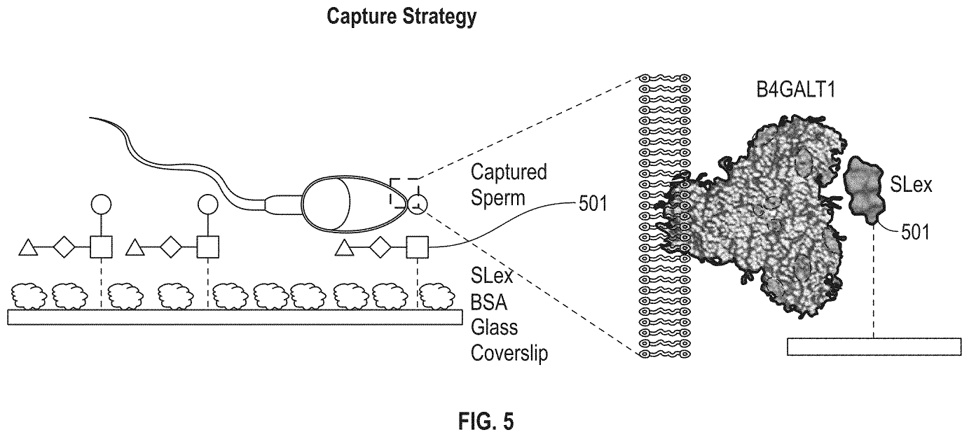

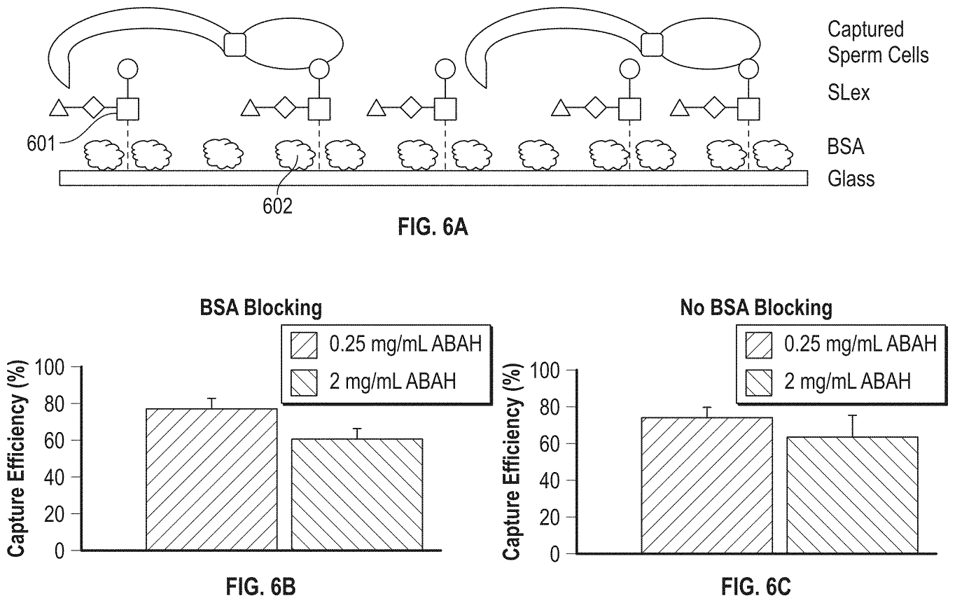

In some embodiments, with reference to FIG. 5, the capturing of sperm cells in the capture area 406 of the microchannels 404 may be accomplished via capturing reagents such as, but not limited to, oligosaccharides that may be utilized for the processing of forensic biological samples. An example of such capture reagents is a unique oligosaccharide (i.e., SLeX sequence [NeuAcα2-3Galβ1-4(Fucα1-3)GlcNAc]) 501 located on the extracellular matrix (i.e., zona pellucida (ZP)) of the oocyte. This oligosaccharide sequence is an abundant terminal sequence on human ZP that represents a ligand for human sperm-egg binding. In some embodiments, the oligosaccharide SLeX agent captures sperm cells by binding to the B4GALT1 (beta1-4galactosyltransferace 1) gene on sperm cells. Discussion of SLeX and its role in sperm-egg binding have been presented in the article by Pang et al., entitled “Human Sperm Binding Is Mediated by the Sialyl-Lewis^{x} Oligosaccharide on the Zona Pellucida,” in the publication Science, 333, 1761 (2011), the entire content of which is expressly incorporated by reference herein. In some embodiments, utilizing such capture reagents, a disposable microfluidic chip that can detect and capture sperm cells from unprocessed bodily fluids can be developed. For example, microfluidic channels that are functionalized using salinization-based surface chemistry may contain immobilized SLeX oligosaccharide 501 that can be used to selectively capture sperm cells. SLeX oligosaccharide 501 has a great advantage over antibody-based methods in that it possesses long shelf life and storage capability. Thus, the disclosed separation and detection platform can allow efficient separation of sperm cells from epithelial cells in sexual assault evidence materials, reducing analysis time and accelerating the forensic process. Specific sperm capture can also be achieved through the use of antibodies, as discussed below with reference to FIG. 6, for example.

In some embodiments, other mechanisms that facilitate the formation of sperm-egg fusion may also be used to capture sperm cells. For example, equatorial segment protein 1 (ESP1), a testis specific protein, has been shown to be highly conserved and to play a key structural role during the fusion of a sperm cell with an egg. As such, any molecule on the oocyte that binds to ESP1 can be used as a capture agent in a similar manner as SLeX oligosaccharide. Some discussion of ESP1 and its role in sperm-egg binding have been presented in the article by Suryavathi et al., entitled “Dynamic Changes in Equatorial Segment Protein 1 (SPESP1) Glycosylation During Mouse Spermiogenesis,” in the publication Biology of Reproduction, vol. 92, no. 5, 129 (2015), the entire content of which is expressly incorporated by reference herein.

In some embodiments, with reference to FIG. 6A, the immobilization of the SLeX oligosaccharide 601 and/or antibodies in the microfluidic channels so as to facilitate the capture of sperm cells may be enhanced by a modified support surface. For example, to link the one or more surface recognition elements such as specific saccharide units, antibodies, etc., into the microchannels, a modified support surface may be formed by a 3-mercaptopropyl-trimethoxysilane (3-MPS) to form thiol groups, reacting N-(gammamaleimidobutyryloxy) succinimide ester (GMBS) with the succinimdie groups to form an amine reactive intermediate, and stabilizing the amine reactive intermediate by 4-Aminobenzoic acid hydrazide (ABAH) to form the modified support surface. For example, a glass slide can be modified with oxygen plasma (100 mW, 1% oxygen) for about 90 seconds in a PX-250 chamber, followed by a silanization step using about 200 mM of 3-mercaptopropyl-trimethoxysilane (3-MPS) dissolved in ethanol. After the silanization step, the glass slide can be assembled with a PMMA-DSA construct to form a microfluidic channel. Further, N-(gammamaleimidobutyryloxy) succinimide ester (GMBS) can be used as an amine reactive intermediate, and after GMBS incubation, the surfaces can be stabilized using an 4-Aminobenzoic hydrazide (ABAH) to create binding groups for SLeX. In some embodiments, to minimize the unspecific binding of cells, a blocking agent 602 may also be employed into the microchannels. The modified support surface may be linked to a SLeX material 601 for the capture of sperm.

For example, with reference to FIGS. 6B and 6C, in some embodiments, the results of experiments conducted where a blocking agent (e.g., Bovine serum albumin (BSA), about 1%) was applied into the microchannels to minimize unspecific binding of cells, and incubated for 30 minutes at 4° C. are shown. Before sampling sperm cells, the channels were washed out with PBS a few times again (e.g., about three times). After surface chemistry steps, sperm samples (˜1,000-5,000 cells/mL) were applied into the channels, and incubated for about 30 minutes at room temperature, followed by microscopy imaging to quantify the sperm cell number in the microchannels. Further, the microchannels were washed with PBS using a syringe pump at about 54/min for about 20 min, followed by microscopy imaging to evaluate the capture efficiency. In the experiments, two different SLeX concentrations and the effect of BSA blocking were evaluated. As a result, it was observed that 0.25 mg/mL of SLeX concentration provided statistically more sperm cell capture in microchannels (n=3-4, p<0.05), e.g., FIG. 6B. The BSA blocking step did not significantly change the capture efficiency of sperm cells when different concentrations were used, e.g., FIG. 6C. In some embodiments, a SLeX modified microfluidic chip may have approximately 77% capture efficiency for human sperm cells by coupling of 4-Aminobenzoic acid hydrazide (ABAH) with this specific carbohydrate unit.

For antibody-based capture events, NeutrAvidin protein, Protein A/G or Protein G may be used to immobilize specific antibodies. Additionally, other or additional antibodies may be present based on the sperm types to be detected. It is contemplated that multiple sperm types may be detected on a single platform. In some embodiments, the antibody may be a polyclonal or monoclonal antibody. Additionally, in some forms, the modified support surface is linked to at least one of a protein A, a protein G, a protein A/G, a Streptavidin protein, and a NeutrAvidin protein which is used to form chemical bonds and as well as physical adsorption to immobilize recognition elements such as the antibody on the modified support surface.

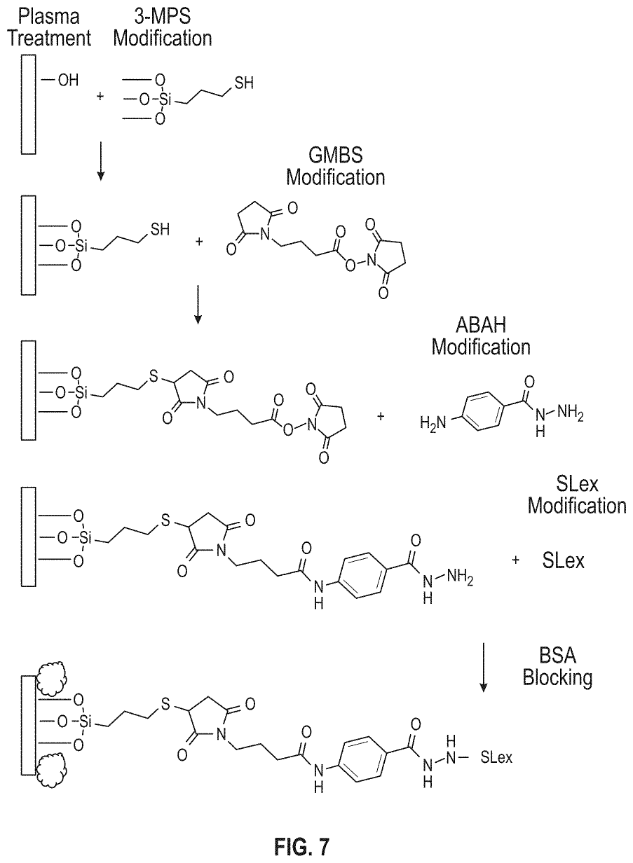

With reference to FIG. 7, in some embodiments, an example process of modifying the surface of one or more channels in the microfluidic cartridge/chip (e.g., with one or more chemicals. and SLeX molecules) to capture sperm cells is shown as an example. Accordingly, after plasma treatment and chip construction, 3-mercaptopropyl-trimethoxysilane (3-MPS) (e.g., about 200 mm in ethanol, about 100 μL) was passed through the channels and incubated for a period of time (e.g., in some embodiments, about 30 minutes) at room temperature. N-(gammamaleimidobutyryloxy) succinimide ester (GMBS) (e.g., about 4% in ethanol, about 30 μL) was then incubated in microchannels for about 45 minutes at room temperature. Hence, GMBS generated succinimide groups to bind amine-functionalized groups, ABAH molecule in this case. Between the chemical modification steps, the channels were washed out with ethanol and PBS (about 100 μL) to remove the excess of untreated reagents. To immobilize the sperm recognition element, two different concentrations of ABAH molecule (about 0.25 mg/mL and about 2.5 mg/mL) were evaluated by incubating for an hour at room temperature. The microchannels were then washed three times with PBS (about 100 μL), followed by an incubation of SLeX solution (about 100 μg/mL in PBS) overnight at 4° C. Then, the microchannels were washed with PBS three times again.

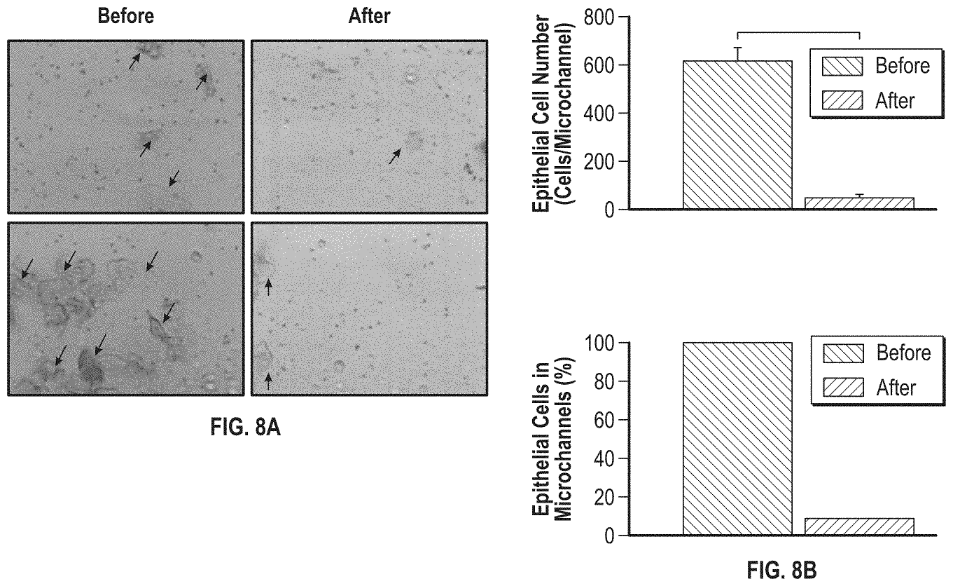

In some embodiments, with reference to FIG. 8, if desired, the sample may be eluted from the capture agents immobilized on the chip to recover the forensic evidence, or in the case of sperm cells, the cells may be lysed (for example, with enzymes and reducing reagents) and DNA recovered for further analysis such as downstream genomic analyses. Other biological entities such as epithelial cells will flow through the chip, and they can be recovered for further processing. Differential extraction processes may be used to allow for the extraction of DNA material from the sperm cells, and if needed from the epithelial cells, with little or no mixing between the DNAs from the different types of cells (i.e., with little or no mixing between the sperm cell DNA and the epithelial cell DNA). See, e.g., K. M. Horsemen, et al., “Separation of Sperm and Epithelial Cells in a Microfabricated Device: Potential Application to Forensic Analysis of Sexual Assault Evidence,” Anal. Chem. 2005, 77, pp. 742-749. For example, the lysis of sperm cells as described above may break down the membranes of the sperm cells, allowing for the extraction of DNA within. For example, chemicals such as dithiothreitol (DTT) may be used to disrupt the sulfur bonds in the coating of sperm cells, facilitating the extraction of the DNA. In some embodiments, standard DNA extraction techniques such as phenol/chloroform extraction and the like may then be utilized.

The shadow imaging platform may provide valuable information to the forensic analyst by quantifying sperm cells from the forensic sample, e.g., FIG. 8B. For example, samples containing 30-40 or more cells (approximately 90-120 pg DNA) may be targeted for standard short tandem repeat (STR) process with commercial kits while samples with less than 20-30 cells may be directed for less informative Y-STR analysis. Also, by comparing the sperm counts from multiple samples, the technology may be used to help direct the analyst to the most probative samples for investigation.

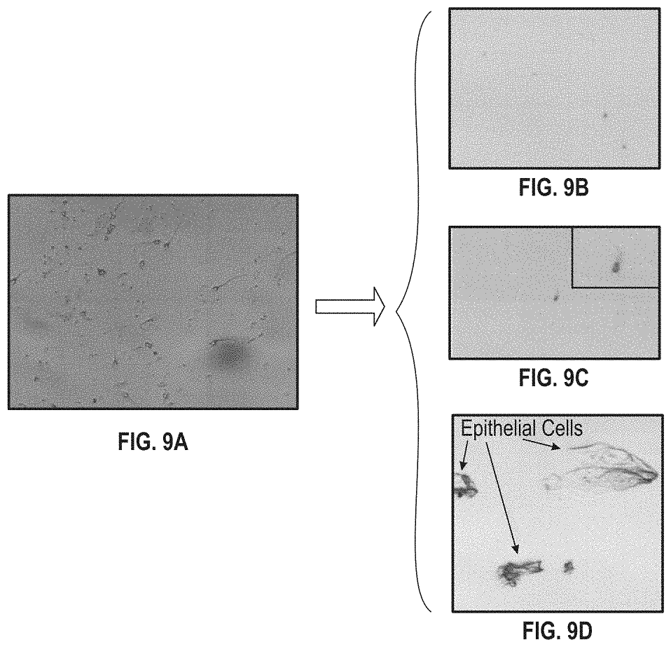

In some embodiments, FIG. 9 shows an example differential extraction process of aged sperm cells from epithelial cells as a demonstration of the capabilities of the platforms and systems disclosed herein. One notes that when aged sperm cells (FIG. 9A) are extracted from epithelial cells (FIG. 9D), most of them have some deformities such as missing tails (FIG. 9B) while a few of them retain their tails (FIG. 9C). Table 1 here shows the results of a differential extraction of aged forensic samples done using the apparatus, systems and methods of the present disclosure. The table shows that using the microfluidic platform of the present disclosure, in some embodiments, a high sperm capture efficiency may be obtained for a variety of samples. In some cases, the impurity level, i.e., the presence of epithelial cells, may be kept to a low level. Sperm capture efficiency may be defined as the ratio of the number of sperm remaining after washing of the sample (i.e., the captured sperm) compared to the number before the capture. In some instances, the impurity level can be measured by the ratio of the number of epithelial cells remaining after washing compared to the initial number of sperm cells, i.e., the number of pre-wash sperm cells. In the specific embodiment of Table 1, the capture efficiency for the samples described therein ranges from about 70% to about 93% while the impurity level ranges from about 6% to about 16%.

TABLE 1

| | | | | Number of | Capture | |
|---|---|---|---|---|---|---|
| | | Isolation/ | Number of | Retained | Efficiency | |
| | Sample | Collection | Captured | Epithelial | of Sperm | Impurity |
| Sample | Explanation | Material | Sperm | Cells | (%) | (%) |
| A | Post coital vaginal | ~⅓ of a | 685 ± 101 | 41 ± 11 | 92.4% | 6.1% |
| | swab with semen | cotton swab | | | | |
| B | Unknown Sample | Cotton gauze | 363 ± 136 | 55 ± 36 | 82% | 16.1% |
| C | Buccal cells mixed | N/A | 275 ± 52 | 58 ± 15 | 82% | 19.8% |
| | with semen | | | | | |
| E | Mixed semen on | Cotton swab | 661 ± 315 | 31 ± 19 | 86.3% | 6.7% |
| | cotton swab | | | | | |
| F | Mixed semen on | Cotton gauze | 289 ± 19 | 48 ± 33 | 70.1% | 16.1% |
| | cotton gauze | | | | | |

Thus, the microfluidics and shadow imaging (for example) means embodiments disclosed herein integrate multiple steps on a single device (e.g., compact, mobile device), improve the scaling capacity, which enables minimal reagent consumption, reducing the need for skilled analysts, etc. Such microfluidic-based embodiments incorporate flow and detection capabilities including optical, electrical and/or mechanical tools for the capture and sort of various type of cells and pathogens (e.g., sperm, white blood cells, bacteria, yeasts, fungi, microbes, and viruses) may be applied to problems of forensic investigation, among other applications.

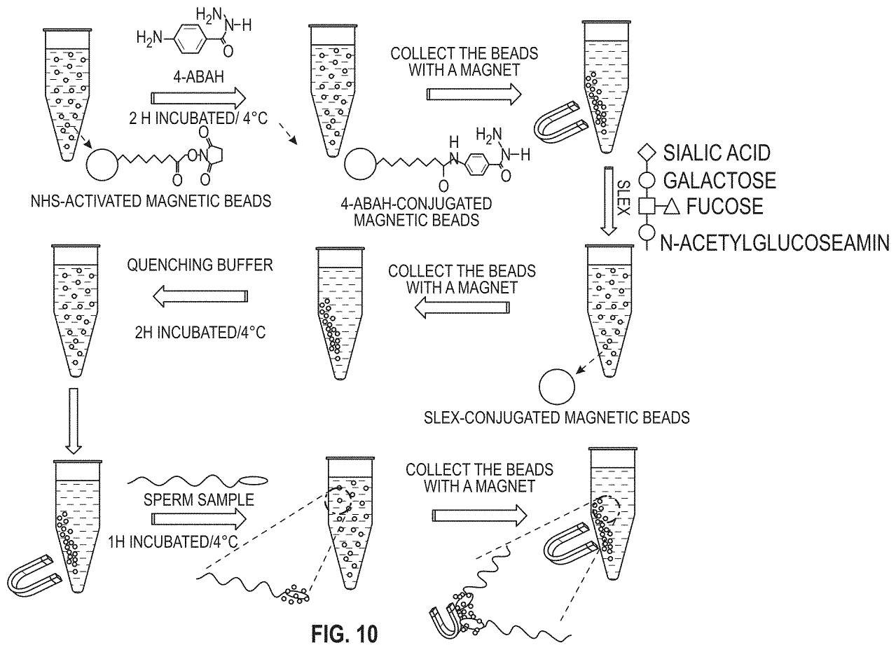

With reference to FIG. 10 shown therein is a flow diagram depicting a non-limiting embodiment of an exemplary process for isolating sperm cells utilizing SLeX conjugated magnetic beads. Initially, surface functionalization of magnetic beads may be accomplished by transferring a homogenous solution of NHS-terminated magnetic beads to a biological sample receptacle, such as a tube, well, cuvette, vial, and/or capillary, and then treating the tube with a magnetic source, such as a magnetic stand, such that the magnetic beads may be collected and the supernatant discarded. The magnetic beads may also be used in conjunction with microfluidic devices. The tube may then be treated with an ice-cold wash buffer, such as 1 mM hydrochloric acid, and vortexed gently before collecting the magnetic beads and discarding the supernatant by using the magnetic stand. The surface of the magnetic beads may be functionalized by forming a modified support surface by utilizing, for example, a recognition reagent such as 4-ABAH to form hydrazide groups on the bead surface. A stock concentration of 4-ABAH may be prepared in DMSO, and adjusted to appropriate concentrations. The 4-ABAH solution may be added to the tube and incubated, for example, for 2 hours on a rotator at 4° C. At the end of the incubation period, the beads can be collected using the magnetic stand and the supernatant can be discarded. The magnetic beads may be washed with, for example, PBS to remove any chemical residue in the tube. After, SLeX solution may be added to the tube and be allowed to incubate, for example, overnight on a rotator at 4° C. Later, the magnetic beads may be washed with DI water. After the washing step, a quenching buffer (including, by way of example only, and not by of limitation, a quenching buffer comprising and/or consisting of 3M of ethanolamine, pH 9.0) may be added to the solution and vortexed. Later, purified water may be added to the tube and mixed well. After, the beads may be collected using the magnetic stand and the supernatant may then be discarded. Then, target cells, such as sperm cells, may be transferred into a tube commonly known in the art and incubated for predetermined amount of time, for example, for an hour on a rotator at 4° C. Following incubation, the beads may be then be collected again using the magnetic stand and the supernatant discarded or transferred elsewhere.

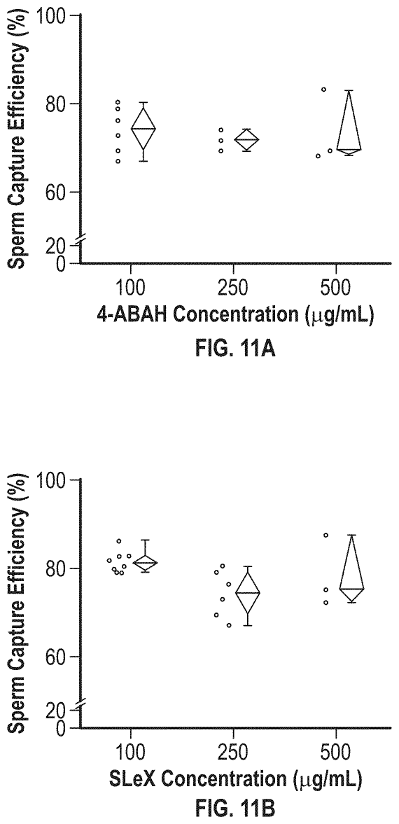

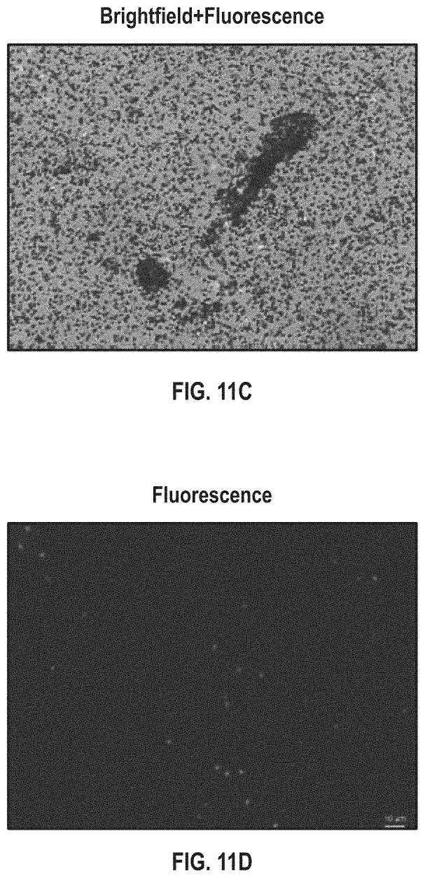

Referring now to FIG. 11A, shown therein are diamond-shaped Box-Whisker plots depicting the results of experiments conducted where sperm capture efficiency was calculated for increasing concentrations of a recognition reagent, such as 4-ABAH. SLeX concentration may be held constant at a predetermined concentration, for example, 250 μg/ml and sperm count number held constant at a predetermined number, for example, 1.5×10⁶ cells in 2004, of sample. A stock concentration of 4-ABAH may be prepared, for example, as 10 mg/ml in DMSO, and experimental concentrations may be adjusted to varying concentrations, for example, 100 μg/ml, 250 μg/ml, and 500 μg/ml with DMSO:PBS (1:1(v:v) ratio). 4-ABAH solutions may be introduced into biological sample receptacles, such as tubes, containing magnetic beads, and incubated for a predetermined time, for example, 2 hours on a rotator at 4° C. After the incubation period, the beads may be collected using a magnetic source, such as a magnetic stand, and the supernatant can be discarded. The magnetic beads may be washed with, for example, PBS to remove any chemical residues in the tube. After the remaining surface chemistry steps, target cells, such as sperm cells, may be added to the tube, and incubated for a predetermined amount of time, for example, 1 hour at a rotator at 4° C. Following incubation, the beads may be collected using the magnetic stand and the supernatant and unbound cells may be transferred into another tube commonly known in the art. Later, the captured sperm cells may be washed with, for example, PBS and the tube may again be placed on the magnetic stand to collect the beads. The unbound cells in the supernatant phase may be quantified using a hemocytometer/cell counter. Captured sperm cell number may be calculated by subtracting the number of cells in the supernatant phase from the stock cell number (sperm count before capture process—such as, by way of example only, and not by of limitation-1.5×106 cells in 200 μL of sample. Sperm capture efficiency rate may be calculated by dividing the sperm count after the capture process by the sperm capture count before the capture process, and multiplying the quotient by 100. In the experiments, three different 4-ABAH concentrations and the effects of sperm capture efficiency were evaluated. As a result, it was observed that there was no statistical difference between the concentrations (n=3-6, p>0.05).

With reference to FIG. 11B, shown therein are diamond-shaped Box-Whisker plots representing the results of experiments where sperm capture efficiency was calculated for increasing concentrations of SLeX. Varying concentrations of SLeX including, by way of example only, and not by of limitation, concentrations of 100 μg/ml, 250 μg/ml, and 500 μg/ml may be applied to the magnetic beads. After the surface chemistry steps, sperm cells, for example, 1.5×10⁶ cells in 200 μL of sample, may be added to the tube, and incubated for a predetermined amount of time, for example, one hour on a rotator at 4° C. In the experiments, three different SLeX concentrations and the impact on sperm capture efficiency were evaluated. As a result, it was observed that there was no statistical difference between all SLeX concentrations over sperm capture efficiency (n=3-8, p>0.05).

FIGS. 11C-11D show fluorescence microscope images of isolated sperm cells stained with DAPI, visible under a fluorescence microscope, as a demonstration of the capture capabilities of modified magnetic beads constructed in accordance with the presently disclosed and/or claimed inventive concept(s).

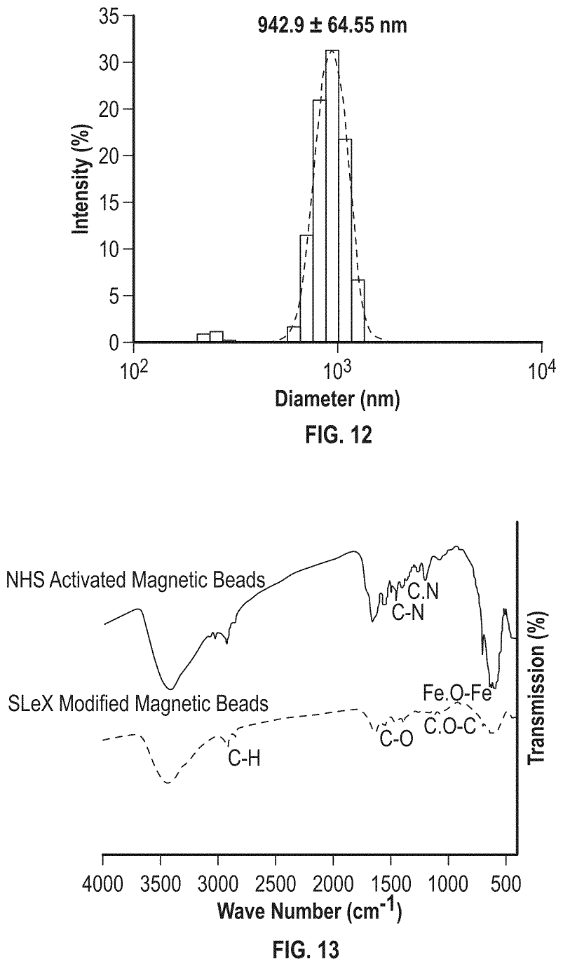

With reference to FIG. 12, shown therein are bar graphs showing example results of the dynamic light scattering (DLS) properties and measurements associated with the particle size of magnetic beads constructed in accordance with the presently disclosed and/or claimed inventive concept(s). It was observed that the magnetic beads possessed mostly monodispersed characteristics with a diameter of 942.9±64.55 nm.

FIG. 13 shows a graphical representation of the results of FTIR spectroscopy measurements of bare magnetic beads (NHS-labeled) and modified beads (including SLeX binding). By comparing the FTIR spectra of both bare and modified beads, it was observed that asymmetric and symmetrical carbonyl stress vibrations caused by NHS groups were reduced. It was further observed that peaks originating from aliphatic groups were more pronounced, indicating that SLeX modification on the NHS-activated magnetic beads were performed successfully.

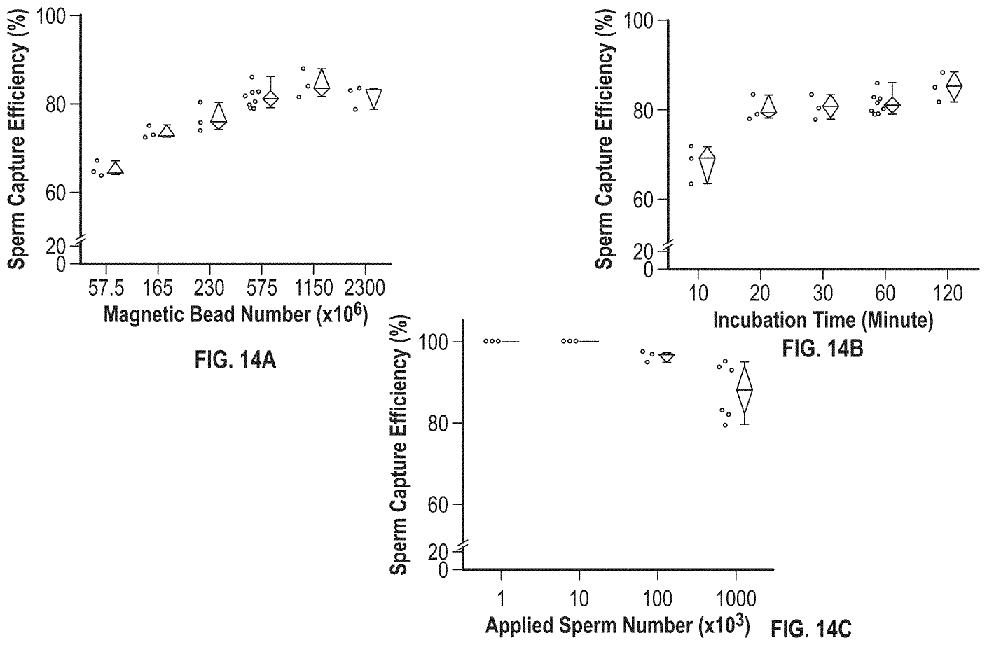

With reference to FIG. 14A, diamond-shaped Box-Whisker plots showing sperm capture efficiency for increasing magnetic bead counts are shown. In the experiment, six different magnetic bead counts and the impact on sperm capture efficiency were evaluated. As a result, it was observed that sperm capture efficiency increased proportionate to bead count, indicating more binding surfaces for sperm capture. It was observed that there was no statistical significance between 575×106, 1,150×106, and 2,300×106 beads (n=3-8, p>0.05).

With reference to FIG. 14B, diamond-shaped Box-Whisker plots depicting sperm capture efficiency for increasing sperm incubation times are shown. In the experiment, five different incubation times and the impact on sperm capture efficiency were evaluated. Sperm capture efficiency may be tested for at various incubation periods, for example, 10, 20, 30, 60, and 120-minute incubation periods. As a result, it was observed that 10 minutes of incubation time resulted in statistically lower sperm capture efficiency than all incubation time slots (n=3-8, p<0.05).

With reference to FIG. 14C, diamond-shaped Box-Whisker plots showing sperm capture efficiency for increasing sperm cell counts are shown. In the experiment, sperm capture efficiency was calculated for when low sperm numbers and high sperm numbers were applied. As a result, it was observed that highest levels of sperm capture efficiency was observed when sperm cells between 1×103 and 10×103 were applied. It was further observed that efficiency decreased starting at 100×103 of sperm cells, and then saturated at the levels from 1,500×10³ to 2,000×10³ cells. It was also observed that lower sperm numbers (1×10³ and 100×10³ cells) provided greater efficiency compared to higher sperm numbers (1,000×10³ and 2,000×10³ cells) when applied (n=3-6. P<0.05). It was further observed that sperm numbers of 100×10³ and 1,000×10³ were statistically similar (n=3-6, p>0.05).

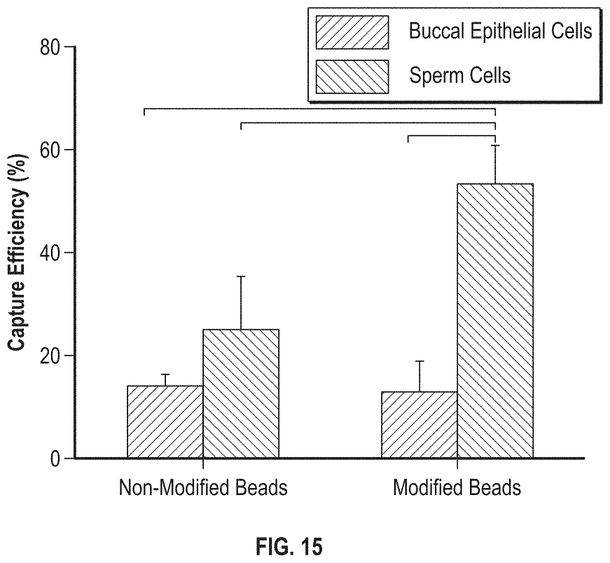

With reference to FIG. 15, a comparative bar graph showing the results of experiments where specificity of modified magnetic beads was calculated is shown. Buccal epithelial cells and sperm cells may be utilized, and a control group, such as for example, non-modified beads may be utilized. This experiment evaluated capture efficiency when utilizing a predetermined magnetic bead count, for example, 287×10⁶ beads and a predetermined incubation time, for example, of 15 minutes. In both experimental conditions (modified and non-modified beads), it was observed that a small portion (8.33% to 16.67%) of buccal epithelial cells remained in the tube, and high number of cells were removed from the solution. As for sperm cell capture, when non-modified beads were applied, it was observed that a small portion (24.58%) of sperm cells remained in the tube whereas the modified beads increased the efficiency to 54.4% (n=2-3, p<0.05), indicating that surface chemistry was critical for sperm capture with high efficiency. Capture efficiency may be increased by the addition of, by way of example only, and not by of limitation, detergents, ionic reagents and/or anti-fouling reagents such as proteins (e.g., bovine serum albumin, casein, glycine, and gelatin), and chemical linkers (e.g., thiol-linkers).

Statistical Analysis.

To evaluate ABAH concentrations, embodiments of the disclosure can employ one-way analysis of variance (ANOVA) with Tukey's posthoc test followed with Bonferroni's Multiple Comparison Test for equal variances for multiple comparisons with a statistical significance threshold set at 0.05 (p<0.05). Error bars in the plots represented standard error of the mean (SEM), and brackets demonstrated the statistical difference between the groups. GraphPad Prism (Version 5.04) was used in all statistical analyses.

Although the above discussion has been provided with respect to a microfluidic device, in some embodiments, all the features of the present disclosure, including the usage of the oligosaccharide SLeX sequence to capture and isolate sperm cells can be applied to non-microfluidic devices. For example, a well-type surface incorporating the oligosaccharide can be used for similar purposes of isolating and capturing biological materials such as sperm cells from biological samples. However, it is worth noting that the surface can be any geometry including a spherical surface (for example), and/or other 2D and 3D geometrical surfaces configured to capture a target (e.g., beads, magnetic beads).

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Some embodiments of the present disclosure may be distinguishable from one and/or another prior art reference by specifically eliminating one and/or another structure, functionality and/or step. In other words, claims to some embodiments of the inventive subject matter disclosed herein may include negative limitations so as to distinguish from the prior art.

When describing the sperm capture platform and binding of an antibody or other molecule thereto (in accordance with the various disclosed embodiments), terms such as linked, bound, connect, attach, interact, and so forth should be understood as referring to linkages that result in the joining of the elements being referred to, whether such joining is permanent or potentially reversible. These terms should not be read as requiring the formation of covalent bonds, although covalent-type bond might be formed.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### Experimental Results Related to Sperm Capture Efficency

Surface Functionalization of Magnetic Beads:

A homogenous solution of NHS-terminated magnetic beads were transferred to an eppendorf tube, and the tube was treated with a magnetic stand. The magnetic beads were then collected, and the supernatant discarded. An ice-cold wash buffer (1 mM of hydrochloric acid) was applied to the tube, and vortexed gently for 15 seconds to mix homogenously. The magnetic beads were collected, and the supernatant discarded. Later, 4-ABAH solution was added to form hydrazide groups on the bead surface. A stock concentration of 4-ABAH was prepared as 10 mg/ml in DMSA, and then adjusted to 100 μg/ml-500 μg/ml with DMSO:PBS (1:1 (v:v) ratio). After adding the 4-ABAH solution to the tube, the tube was incubated for 2 hours on a rotator at 4° C. (during the first 30 minutes, the tube was vortexed for 15 seconds at every 5 minutes). At the end of the incubation period, the beads were collected using the magnetic stand, and the supernatant was discarded. The magnetic beads were then washed with PBS to remove any chemical residue (2 times).

A SLeX solution (100 μg/ml-500 μg/ml in PBS) was added into the tube and allowed for an overnight incubation on a rotator at 4° C. (during the first 30 minutes, the tube was vortexed for 15 seconds at every 5 minutes). The magnetic beads were then washed with DI water (2 times). After the washing step Quenching Buffer (3M of ethanolamine, pH 9.0) was added to the bead solution, and the tube was vortexed for 30 seconds. The tube was incubated for 2 hours on a rotator at 4° C. Later, purified water was added to the tube, and the tube was mixed well. At the end, the beads were collected with a magnetic stand, and the supernatant was discarded.

Size Distribution of Magnetic Beads: The size distribution of NHS-activated beads were analyzed with the dynamic light scattering (DLS) system. The size distribution of beads was performed in a standard DLS cuvette. A solution containing 25 μL of bead solution and 2975 μL DI water was made, and that solution was measured in a cuvette at 25° C.

Sperm capture efficiency was measured by altering various variables, such as sample incubation time, number of magnetic beads and sperm cells, and the concentration of 4-ABAH and SLeX solutions. Sperm cell efficiency was measured by dividing sperm count after capture process by the sperm count before the capture process and multiplying that quotient by 100 to obtain an efficiency percentage. Sperm count after the capture process was calculated by subtracting the number of sperm cells in the supernatant phase from sperm count before the capture process. The data was analyzed using a one-way analysis of variance (ANOVA) followed by Tuckey's multiple comparison test.

4-ABAH Concentration Optimization: 4-ABAH concentration over capture efficiency was determined by first preparing a stock concentration of 4-ABAH as 10 mg/ml in DMSO. The stock concentration was adjusted with DMSO:PBS (1:1 ratio) to produce 100 μg/ml, 250 μg/ml, and 500 μg/ml concentration 4-ABAH solution. Sperm capture efficiency was tested at each different concentration of 4-ABAH solution, while holding constant sperm cell count at 1.5×10⁶ cells in 200 μL of sample and SLeX solution at 250 μg/ml. NHS-terminated magnetic beads were treated with each concentration of 4-ABAH solution. The results are listed below in Table 2.

TABLE 2
Sperm Capture Efficiency over 4-ABAH Concentration

| 4-ABAH | Sperm Capture | SLeX | |
|---|---|---|---|
| Concentration | Efficiency | Concentration | Sperm Number |
| (μg/ml) | (%) | (μg/ml) | (cells/200 μL) |
| 100 | 74.3 ± 5.4 | 250 | 1.5 × 10⁶ |
| 250 | 71.8 ± 2.4 | 250 | 1.5 × 10⁶ |
| 500 | 73.7 ± 8.5 | 250 | 1.5 × 10⁶ |

SLeX Concentration Optimization: SLeX concentration over capture efficiency was determined by adjusting concentrations of SLeX solution while holding 4-ABAH concentration constant at 100 μg/ml, and sperm cell count at 1.5×10⁶ cells in 200 μL of sample. SLeX solution was adjusted to concentrations of 100 μg/ml, 250 μg/ml, and 500 μg/ml. Sperm capture efficiency was tested at each different concentration of SLeX. The results are listed below in Table 3.

TABLE 3
Sperm Capture Efficiency over SLeX Concentration

| 4-ABAH | Sperm Capture | SLeX | |
|---|---|---|---|
| Concentration | Efficiency | Concentration | Sperm Number |
| (μg/ml) | (%) | (μg/ml) | (cells/200 μL) |
| 100 | 81.6 ± 2.4 | 100 | 1.5 × 10⁶ |
| 100 | 74.3 ± 5.4 | 250 | 1.5 × 10⁶ |
| 100 | 78.2 ± 8.2 | 500 | 1.5 × 10⁶ |

Magnetic Bead Count Optimization: Magnetic bead count over capture efficiency was determined by adjusting magnetic bead count from the stock solution to concentrations of 57.5×10⁶, 165×10⁶, 230×10⁶, 575×10⁶, 1,150×10⁶, and 2,300×10⁶ beads. Sperm capture efficiency was tested at each set count of magnetic beads. It was observed that sperm capture efficiency increased proportionate to the bead count. The results are listed below in Table 4.

TABLE 4
Sperm Capture Efficiency over Magnetic Bead Count

| Sperm Capture Efficiency | Magnetic bead count |
|---|---|
| (%) | (beads) |
| 65.2 ± 1.8 | 57.5 × 10⁶ |
| 73.7 ± 1.4 | 165 × 10⁶ |
| 76.8 ± 3.3 | 230 × 10⁶ |
| 81.6 ± 2.7 | 575 × 10⁶ |
| 84.5 ± 3.2 | 1,150 × 10⁶ |
| 81.8 ± 2.7 | 2,300 × 10⁶ |

Sperm Incubation Time Optimization: The effect of sperm incubation time over capture efficiency was determined by altering incubation periods spanning from 10 minutes to 120 minutes. It was observed that sperm capture efficiency increases incrementally when increasing incubation time Sperm capture efficiency was tested at incubation times of 10, 20, 30, 60, and 120 minutes. The results are listed below in Table 5.

TABLE 5
Sperm Capture Efficiency over Incubation Time

| Sperm Capture Efficiency | Sperm incubation time |
|---|---|
| (%) | (minutes) |
| 68.2 ± 4.4 | 10 |
| 80.3 ± 2.9 | 20 |
| 80.7 ± 2.8 | 30 |
| 81.6 ± 2.4 | 60 |
| 85.2 ± 3.4 | 120 |

Sperm Cell Count Optimization: The effect of the number of sperm cells over capture efficiency was examined by applying a varying number of sperm cells. It was observed that sperm capture efficiency was dependent on sperm number where magnetic bead count was held constant. Sperm capture efficiency was tested at sperm cell counts of 1×10³ to 100×10³ cells and 1,000×10³ to 2,000×10³ cells. It was observed that sperm capture efficiency is highest when sperm cells were applied at a number between 1×10³ and 10×10³ cells. The efficiency decreased starting from 100×10³ cells, and then saturated at the levels from 1,500×10³ to 2,000×10³ cells. The lower sperm numbers (1×10³ to 100×10³ cells) provided greater efficiency compared to the higher sperm numbers (1,000×10³ to 2,000×10³ cells) applied to the tube (n=3-6, p>0.05). There was no statistical significance observed between the lower sperm numbers (n=3-6, p>0.05), and similarly there was no difference observed between the higher sperm numbers (n=3-6, p>0.05). Further, it was observed that the data derived from sperm numbers of 100×10³ and 1,000×10³ was statistically similar (n=3-6, p>0.05). Sperm samples were stained with DAPI, and counted using a hemocytometer/cell counter.

Characterization of Magnetic Beads and Surface Chemistry: Magnetic beads were first characterized with their size parameter using dynamic light scattering (DLS) measurements. The meads had mostly monodispersed characteristics with a diameter of 942.9±64.55 nm. The surface chemistry was characterized by comparing the chemical functionality of bare magnetic beads (NHS-labeled) and modified beads (including SLeX binding). In the FTIR spectrum of bare beads, a characteristic peak of 630 cm⁻¹ of Fe—O—Fe was observed. The other observed peak was Fe—O tensile vibration at 440 cm⁻¹. In addition to iron oxide peaks, succinimide units on the bare beads were clearly visible. Briefly, the asymmetric and symmetric imide stretching vibrations at ˜1718-1750 cm⁻¹ and C—C stretching vibration at 1250 cm⁻¹ were observed. The tensile vibration of 1453 cm⁻¹ C—N was also clearly visible over imide groups. In the SLeX modified beads, the characteristic peaks of the SLeX were observed at 2850-2925 cm⁻¹ (C—H stretching frequency) and ˜1610-1650 cm⁻¹ (C═O stretching frequencies). Besides, the C—O—C etheric tensile vibration appeared at 1066 cm⁻¹.

The chemical structure of the NHS-activated magnetic beads and SLeX-activated magnetic beads was characterized using the Fourier Transformed Infrared (FTIR) system (PerkinsElmer 283 spectrum). A 4 mg sample was pelleted with KBr, and the FTIR spectra was recorded in the wave length range of 400-4,000 cm⁻¹.

### Example 1: Buccal Epithelial Cells

Modified magnetic beads with a count of 287×106 beads were placed into a microcentrifuge tube. 4-ABAH solution with a concentration of 100 μg/ml was added into the microcentrifuge tube. The magnetic beads were collected and the supernatant was discarded after applying a magnet to the microcentrifuge tube. A SLeX solution with a concentration of 100 μg/ml was added into the tube and allowed for incubation overnight. The beads were again collected and the supernatant was discarded after applying a magnet to the tube. Quenching buffer was added (3 M of ethanolamine, pH 9.0) to the tube and was allowed to incubate for 2 hours. The beads were collected and the supernatant was discarded again after applying a magnet to the tube. Buccal epithelial cells were added into the tube, and incubated for 15 minutes. The beads were then collected and the supernatant was discarded after applying a magnet to the tube. Non-modified beads were used as controls. The data was analyzed through a one-way ANOVA test followed by Tukey's multiple comparison test. It was observed, with both modified and non-modified beads, that 8.33% to 16.67% of buccal epithelial cells remained in the tube, with the remainder removed from the solution. The sperm capture efficiency of the non-modified beads was observed to be 24.58%, whereas the efficiency of the modified beads was observed at 53.3% (n=2-3, p<0.05). It was observed that specificity was improved with decreasing bead number and incubation time.

### Example 2: Preparing Vaginal Epithelial Cells

Primary Vaginal Epithelial Cells (ATCC® PCS-480-010™) from ATCC were used. Briefly, the cryovial containing vaginal epithelial cells were thawed rapidly in a 37° C. water-bath. The cell suspension in the cryovial was slowly transferred to a tube containing 9 mL of medium (Vaginal Epithelial Cell Basal Medium (ATCC® PCS-480-030™)), and then, the cell suspension was centrifuged at 1500 rpm for 3 minutes. Afterwards, the supernatant was discarded and 1 mL of medium was applied to the pellet. Later, 100 μL of cell suspension was pipetted in cell culture dishes containing 5 mL of growth media and the dishes were placed in a 5% CO₂ incubator at 37° C. The dishes were checked daily using an inverted microscope and the cell confluence was monitored regularly. The medium was then removed from primary culture using a sterile pasteur pipet and the adhering cells were washed once with PBS. The trypsin/EDTA solution was added to culture in order to remove adhering cell layer. Then the dishes were placed in a 5% CO₂ incubator at 37° C. for 3 minutes. The culture was then checked again using an inverted microscope to ensure the cells detached from the surface. The cell suspension was transferred to a tube containing 2 mL of culture medium. The tube was then centrifuged at 1500 rpm for 3 minutes and the supernatant was discarded. Finally, 1 mL of medium was applied to the pellet and vaginal epithelial cells were counted on a hemocytometer.

### Non-Limiting Examples of Presently Disclosed Inventive Concept

A system for detecting and isolating target cells in a biological sample, the system comprising a biological sample receptacle configured to receive a biological sample, wherein the biological sample receptacle contains one or more magnetic beads, wherein the magnetic beads comprise at least one outer surface, further wherein the biological sample receptacle comprises at least one outer surface and at least one inner surface; wherein a recognition reagent is linked to the outer surface of the one or more magnetic beads, wherein the recognition reagent is configured to capture one or more target cells contained in the biological sample by binding with the one or more target cells, and further wherein a magnetic source is configured to apply an external magnetic field to the outer surface of the biological sample receptacle thereby immobilizing the one or more magnetic beads to the at least one inner surface of the biological sample receptacle.

The biological sample receptacle of the system is selected from the group consisting of a well, cuvette, tube, vial, capillary, and combination thereof.

The biological sample receptacle of the system is a microfluidic device.

The one or more target cells of the system is selected from the group consisting of sperm cells, blood cells, bacteria, yeast, fungi, viruses, and combinations thereof.

The one or more target cells of the system are sperm cells.

The recognition reagent of the system comprises a saccharide sequence.

The saccharide sequence of the system comprises an oligosaccharide sequence.

The oligosaccharide sequence of the system comprises a sialyl-Lewis^{x} oligosaccharide sequence.

A method for detecting and isolating target cells in a biological sample, the method comprising the steps of providing a biological sample receptacle, the biological sample receptacle having at least one outer surface and at least one inner surface, wherein the biological sample receptacle is configured to accept the biological sample, the biological sample comprising one or more target cells; transferring one or more magnetic beads having at least one outer surface, into contact with the at least one inner surface of the biological sample receptacle, the at least one outer surface of the one or more magnetic beads being linked to a recognition reagent, wherein the recognition reagent is configured to capture the one or more target cells; exposing the biological sample to the at least one outer surface of the one or more magnetic beads; capturing the one or more target cells contained in the biological sample, wherein the one or more target cells are captured by binding with the recognition reagent; and applying an external magnetic source to the at least one outer surface of the biological sample receptacle to immobilize the one or more magnetic beads to the at least one inner surface of the biological sample receptacle.

The biological sample receptacle of the method is selected from the group consisting of a well, cuvette, tube, vial, capillary, and combination thereof.

The biological sample receptacle of the method is a microfluidic device.

The one or more target cells of the method are selected from the group consisting of sperm cells, blood cells, bacteria, yeast, fungi, viruses, and combinations thereof.

The one or more target cells of the method are sperm cells.

The recognition reagent of the method comprises a saccharide sequence.

The saccharide sequence of the method comprises an oligosaccharide sequence.

The oligosaccharide sequence of the method comprises a sialyl-Lewis^{x} oligosaccharide sequence.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A system for detecting and isolating target cells in a biological sample, comprising:
a biological sample receptacle configured to receive a biological sample, the biological sample receptacle containing one or more magnetic beads, wherein the magnetic beads comprise at least one outer surface, further wherein the biological sample receptacle comprises at least one outer surface and at least one inner surface;
a recognition reagent linked to the outer surface of the one or more magnetic beads, the recognition reagent configured to capture one or more target cells contained in the biological sample by binding with the one or more target cells; and
a magnetic source configured to apply an external magnetic field to the outer surface of the biological sample receptacle thereby immobilizing the one or more magnetic beads to the at least one inner surface of the biological sample receptacle.

2. The system of claim 1, wherein the biological sample receptacle is selected from the group consisting of a well, cuvette, tube, vial, capillary, assay strip, and combinations thereof.

3. The system of claim 1, wherein the biological sample receptacle is a microfluidic device.

4. The system of claim 1, wherein the one or more target cells are sperm cells.

5. The system of claim 1, wherein the recognition reagent comprises a saccharide sequence.

6. The system of claim 6, wherein the saccharide sequence comprises an oligosaccharide sequence.

7. The system of claim 7, wherein the oligosaccharide sequence comprises a sialyl-Lewis^{x} oligosaccharide sequence.

8. A method for detecting and isolating target cells in a biological sample, comprising:
providing a biological sample receptacle, the biological sample receptacle having at least one outer surface and at least one inner surface, wherein the biological sample receptacle is configured to accept the biological sample, the biological sample comprising one or more target cells;
transferring one or more magnetic beads having at least one outer surface, into contact with the at least one inner surface of the biological sample receptacle, the at least one outer surface of the one or more magnetic beads being linked to a recognition reagent, wherein the recognition reagent is configured to capture the one or more target cells;
exposing the biological sample to the at least one outer surface of the one or more magnetic beads;
capturing the one or more target cells contained in the biological sample, wherein the one or more target cells are captured by binding with the recognition reagent; and
applying an external magnetic source to the at least one outer surface of the biological sample receptacle to immobilize the one or more magnetic beads to the at least one inner surface of the biological sample receptacle.

9. The method of claim 9, wherein the biological sample receptacle is selected from the group consisting of a well, cuvette, tube, vial, capillary, assay strip, and combinations thereof.

10. The method of claim 9, wherein the biological sample receptacle is a microfluidic device.

11. The method of claim 9, wherein the one or more target cells are sperm cells.

12. The method claim 9, wherein the recognition reagent comprises a saccharide sequence.

13. The method of claim 14, wherein the saccharide sequence comprises an oligosaccharide sequence.

14. The method of claim 15, wherein the oligosaccharide sequence comprises a sialyl-Lewis^{x} oligosaccharide sequence.
